(19) United States
(12) Patent Application Publication
(10) Pub. No.: US 2022/0223231 A1
(43) Pub. Date: Jul. 14, 2022

(54) Systems and Methods for Improved Prognostics in Medical Imaging

(71) Applicant: The Board of Trustees of the Leland Stanford Junior University, Stanford, CA (US)

(72) Inventors: Fabian H. Reith, Stanford, CA (US); Greg Zaharchuk, Stanford, CA (US)

(73) Assignee: The Board of Trustees of the Leland Stanford Junior University, Stanford, CA (US)

(21) Appl. No.: 17/647,950

(22) Filed: Jan. 13, 2022

Related U.S. Application Data
(60) Provisional application No. 63/137,626, filed on Jan. 14, 2021.

(51) Int. Cl. G16B 40/20; G16B 5/20; G06N 20/00; G06N 3/02

(52) U.S. Cl. CPC G16B 40/20; G06N 3/02; G06N 20/00; G16B 5/20

(57) ABSTRACT

Methods and systems for predicting biomarker progression in medical imaging is provided. A predictive model can be utilized to predict progression of a medical disorder as determined by progression of the predicted biomarker. Further, the predicted biomarker progression can be utilized to identify individuals that are fast progressors, moderate progressors, slow progressors. In some instances, the enrollment within clinical trials or treatment regimens are determined based on biomarker progression.

### CROSS REFERENCE TO RELATED APPLICATIONS

This application claims priority to U.S. Provisional Application Ser. No. 63/137,626, entitled “Systems and Methods Using Machine Learning for Improved Prognostics in Medical Imaging,” filed Jan. 14, 2021, which is incorporated herein by reference in its entirety.

### TECHNOLOGY FIELD

The disclosure is generally directed to methods to predict biomarkers in medical imaging and applications thereof.

### BACKGROUND

While correct disease diagnosis is required to select appropriate therapies, knowledge about the disease evolution and prognosis is also critically important. For example, some diseases might be self-limited and require minimal or no interventions on the behalf of treating physicians. Other diseases might progress with faster and more virulent time courses, pushing providers to consider higher-risk, more invasive therapies. Furthermore, prognosis information plays a critical role in patient discussions and resource planning. While radiology has so far focused largely on diagnosis, an opportunity exists to use radiological information to risk stratify patients regarding disease prognosis.

Dementia, such as Alzheimer's disease (AD), is one condition where prognosis is especially important. Understanding the likelihood and rate of progression of this disease would be extremely helpful not only for diagnosing and assessing disease severity in individual patients, but also to plan clinical trials. It is well known that AD clinical trials face significant challenges with enrollment due to the high level of variation in the rate of disease progression. Being able to selectively recruit patients likely to progress quickly, either based on cognitive testing or on brain imaging biomarkers such as amyloid and tau deposition, could significantly impact the design, duration, and cost of clinical trials of new pharmaceuticals.

### SUMMARY

Many embodiments are directed to methods of predicting biomarkers in medical imaging. In many of these embodiments, a trained and validated computational model predicts a future status of biomarkers utilizing a subject's baseline images. Many embodiments utilize the predicted biomarker status to provide diagnostics and treatments.

In an embodiment is a method of predicting future biomarkers. The method obtains a set of one or more baseline medical images. The set of one or more baseline medical images was captured from a subject. The set of baseline medical images contains one or more biomarkers that are associated with a medical disorder. The method utilizes a predictive model and the set of baseline biomedical images to predict the progression of the one or more biomarkers.

In an embodiment is a computational system for predicting biomarkers. The computational system includes a memory, a set of one or more processors, and an application stored the memory. The application is a predictive computational model for predicting biomarkers. The set of one or more processors is capable of performing the steps of the application. The steps include assessing a set of one or more baseline medical images. The set of one or more baseline medical images was captured from a subject. The set of baseline medical images contains one or more biomarkers that are associated with a medical disorder. The steps include predicting the progression of the one or more biomarkers based on the assessment of one or more baseline medical images.

### BRIEF DESCRIPTION OF THE DRAWINGS

The description and claims will be more fully understood with reference to the following figures and data graphs, which are presented as exemplary embodiments of the disclosure and should not be construed as a complete recitation of the scope of the disclosure.

FIG. 1 provides a flow diagram of a method to train a machine learning for predicting future biomarkers from a baseline image in accordance with various embodiments.

FIG. 2 provides a flow diagram of a method to predict future biomarkers utilizing a trained predictive model in accordance with various embodiments.

FIG. 3 provides a schematic of a computational system for predicting future biomarkers in accordance with various embodiments.

FIG. 4A provides a data graph depicting distribution of amyloid PET SUVR values at baseline (n=2577), utilized in accordance with various embodiments. The black line represents the suggested threshold between amyloid negative and positive patients.

FIG. 4B provides a data graph depicting SUVR change over time showing small increases in SUVR generally for baseline amyloid positive subjects compared with amyloid negative subjects, generated in accordance with various embodiments.

FIG. 5 provides a schematic depicting an overview of ResNet-50 training procedure in accordance with various embodiments. Three central slices are fed into the input color channels. The ResNet algorithm is modified to perform regression on SUVR rather than classification. Finally, for the testing of ΔSUVR prediction, 2048 deep features (scalar values) were extracted from the final fully-connected layer (fc1).

FIG. 6 provides a flow diagram of a method showing how different scans were used for training and testing of the algorithms in accordance with various embodiments. Cohorts highlighted in blue were used for training, while those highlighted in green were used for testing.

FIG. 7 provides a schematic showing the three tested models in accordance with various embodiments. Left to right: Multivariate linear regression (8 input features), GBDT based on the 8 clinical features only, GBDT based on clinical features and ResNet activations (8+2048 input features).

FIG. 8 provides data graphs depicting training and test set performance of machine learning models, generated in accordance with various embodiments. Root mean-squared error (RMSE) between prediction and the true ΔSUVR is shown, so lower values represent better model performance, with best performance seen for the GBDT using the image-based activations trained on the larger n=1441 subject dataset.

FIG. 9 provides scatterplots featuring ground truth ΔSUVR and the predictions of the different ML approaches, generated in accordance with various embodiments. Best performance is demonstrated by the GBDT model with deep activations.

FIG. 10 provides data graphs of RMSE for predicting ΔSUVR for varying time frames and for varying patient subsets, generated in accordance with various embodiments. The test set RMSE for predicting ΔSUVR at different time periods after the original scan demonstrates that performance overall decreases for predictions farther in the future and that the GBDT with activations was always the best performing model. The test set RMSE for predicting ΔSUVR in different patient subsets also demonstrates that the GBDT with activations was always the best performing model.

FIG. 11 provides data graphs depicting RMSE performance when missing a particular feature in the model, generated in accordance with various embodiments.

FIG. 12 provides data graphs showing percentage of ground truth top 61 (top 10%) progressors who are also found in top 61 progressors predicted by ML model or random pick, generated in accordance with various embodiments.

FIG. 13 provides data graphs showing percentage of ground truth top 31 progressors also found in top 31 progressors for mildly amyloid positive patients (SUVR between 0.79 and 0.95, n=156) and percentage of ground truth top 46 progressors also found in top 46 progressors for CDR 0.5 subjects (n=229), generated in accordance with various embodiments.

### DETAILED DESCRIPTION

Turning now to the drawings and data, systems and methods for generating deep learning computational models for predicting future biomarkers in medical imaging, in accordance with various embodiments, are provided. Some embodiments are directed towards utilizing medical imaging data to train a predictive computational model to predict future biomarkers based on a single or few baseline images. In some embodiments, the trained predictive computational model is then utilized to predict biomarkers that are likely to develop over time. In many embodiments, the results of predicted biomarkers are used to assess and/or diagnose the progression of a medical disorder of an individual. In some embodiments, the trained predictive computational model is trained to predict development of biomarkers utilizing baseline image biomarker feature data. In some embodiments, a deep learning computational model is utilized to learn important biomarker features to be utilized within the predictive computational model. Various embodiments are directed to the use of various medical imaging modalities, including (but not limited to) positron emission tomography (PET), computed tomography (CT), magnetic resonance imaging (MRI), X-ray, fluoroscopic imaging, and ultrasound sonography as relevant for a particular disease, syndrome, ailment, and/or other medical condition. In some embodiments, imaging modalities are combined, as appreciated in the art. For example, in some embodiments, PET is combined with CT to observe amyloid deposits in known or suspected AD patients.

Dementia is one condition where prognosis is especially important. Understanding the likelihood and rate of progression of this disease would be extremely helpful, not only for individual patients and families, but also to plan clinical trials. Alzheimer's disease (AD) trials face significant challenges with enrollment. (See e.g., Grill J D, Karlawish J. Addressing the challenges to successful recruitment and retention in Alzheimer's disease clinical trials. Alzheimers Res Ther. 2010; 2: 34; and Clement C, et al. Challenges to and Facilitators of Recruitment to an Alzheimer's Disease Clinical Trial: A Qualitative Interview Study. J Alzheimers Dis. 2019; 69: 1067-1075; the disclosures of which are hereby incorporated by reference in their entireties.) Being able to selectively recruit patients likely to progress quickly, based in part on brain imaging biomarkers such as amyloid and tau deposition, could significantly impact the design, duration, and cost of clinical trials.

Deep learning has shown much promise in classifying patients and predicting their future disease trajectories. (See e.g., Ding Y, et al. A Deep Learning Model to Predict a Diagnosis of Alzheimer Disease by Using 18F-FDG PET of the Brain. Radiology. 2019; 290: 456-464; Hekler A, et al. Pathologist-level classification of histopathological melanoma images with deep neural networks. Eur J Cancer. 2019; 115: 79-83; Miotto R, Li L, Dudley J T. Deep Learning to Predict Patient Future Diseases from the Electronic Health Records. Advances in Information Retrieval. Springer International Publishing; 2016. pp. 768-774; and Yoo Y, et al. Deep Learning of Brain Lesion Patterns for Predicting Future Disease Activity in Patients with Early Symptoms of Multiple Sclerosis. Deep Learning and Data Labeling for Medical Applications. Springer International Publishing; 2016. pp. 86-94; the disclosures of which are hereby incorporated by reference in their entireties.) It has also been used at the image level to transform images, either for better image reconstruction or the synthesis of desired contrasts (i.e., predicting CT from MRI to enable MR-based PET attenuation correction). (See e.g., Hammernik K, et al. Learning a variational network for reconstruction of accelerated MRI data. Magn Reson Med. 2018; 79: 3055-3071; Zhu B, Liu J Z, Cauley S F, Rosen B R, Rosen M S. Image reconstruction by domain-transform manifold learning. Nature. 2018; 555: 487-492; and Liu F, et al. Deep Learning MR Imaging-based Attenuation Correction for PET/MR Imaging. Radiology. 2018; 286: 676-684; the disclosures of which are hereby incorporated by reference in their entireties.)

In accordance with several embodiments, one or more features of clinical, genetic, and imaging features of a baseline image are combined and computationally analyzed to predict progression of biomarkers over time. In many embodiments, the predicted biomarkers are then further assessed to identify individuals at highest risk of rapid biomarker progression. For instance, in some embodiments, the quantitative change in amyloid beta protein deposits are predicted and utilized to assess risk of AD development. As described herein, a computational deep-learning model utilizing baseline image features and gradient-boosted random forest regression outperforms other existing methods for predicting biomarker progression. Further, the baseline imaging features are shown to be able to better detect individuals with fast disease progression. In some embodiments, fast progressors are treated more aggressively. In some embodiments, fast progressors are utilized within clinical trials, which can expedite assessment of potential medications and treatments.

### Model Development for Prediction of Future Biomarkers

A number of embodiments are directed to predicting future biomarkers in medical imaging from a set of one or more baseline images. A medical disorder is to be understood to be any physical or mental condition or risk of a physical or a mental condition that can be medically assessed, especially disorders assessable via imaging. In some embodiments, a medical disorder is a deviation of a physical or a mental condition from the norm, which can often result in a physical or mental ailment. In some embodiments, the medical disorder is a neurodegenerative disorder and the biomarker to be predicted is the accumulation of aggregates. In certain embodiments, the medical disorder is Alzheimer's disease and the biomarker to be predicted is accumulation of amyloid beta protein. In certain instances, amyloid beta protein amount is quantified by the standardized uptake value ratio (SUVR), which is determined by positron emission tomography (PET) and the amyloid radiotracer 18F-AV45 (florbetapir).

In some embodiments, a predictive computational model is utilized to predict progression of biomarker over a period of time. Any appropriate machine learning model can be utilized, including (but not limited to) linear regression (e.g., LASSO) and gradient-boosted random forest techniques (e.g., gradient-boosted decision trees). Likewise, any appropriate model architecture can be utilized that provides an ability to predict future biomarkers.

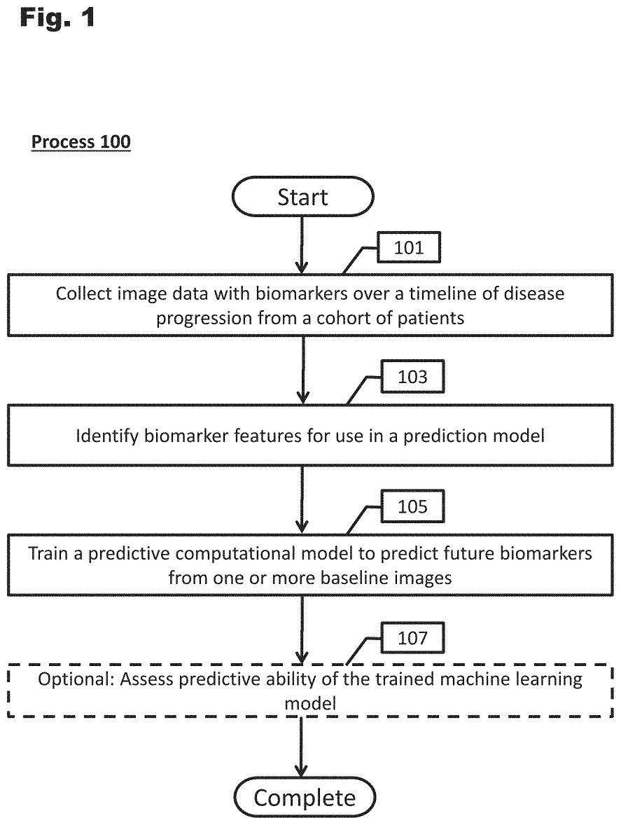

Provided in FIG. 1 is a method to build, train, and assess a predictive model that predicts future biomarkers from one or more baseline images in accordance with various embodiments. Process 100 begins with collecting (101) medical image data of a cohort of patients having a medical similarity (e.g., medical disorder). Typically, the disorder to be modeled progresses in a manner that is assessable via biomarkers of medical imaging. Examples of risks and/or disorders that can be monitored via medical imaging biomarkers include (but are not limited to) neurodegenerative diseases (e.g., Parkinson's disease, Alzheimer's disease). In certain embodiments, the medical disorder is Alzheimer's disease and the biomarker to be predicted is accumulation of amyloid beta protein. In certain embodiments, the medical disorder is Parkinson's disease and the biomarker to be predicted is accumulation of Lewy bodies.

Any appropriate medical image data can be utilized that provides analysis of biomarkers of disorder progress. Likewise, any appropriate imaging modality may be utilized, as appropriate for the disorder and relevant biomarker to be monitored. Examples of medical imaging modalities include (but are not limited to) magnetic resonance imaging (MRI), X-ray, fluoroscopic imaging, computed tomography (CT), ultrasound sonography (US), and positron emission tomography (PET). Various imaging modalities can be combined, such as PET-CT scanning. Likewise, various image data derived from multiple modalities can be collected and be utilized as training data. Further, any appropriate image data can be derived from the collected images and utilized as training data. Images can be acquired by any appropriate means for the disorder to be monitored, including various contrasting methods. Likewise, images can be processed as appropriate. In some embodiments, collected images are normalized between patients within the cohort.

In some embodiments, images are collected from each patient of the cohort over an appropriate period of time. In some embodiments, a baseline image is collected, which is denoted as t=0 for the individual. In some embodiments, images are collected at specific time intervals. In some embodiments, images are collected at specific disorder events. In some embodiments, images are collected until a predesignated endpoint. In some embodiments, images are collected until a medical or terminal event.

As depicted in FIG. 1, biomarker features are identified (103) for use in a prediction model. In some embodiments, a deep learning model is utilized to identify biomarker features from baseline images. In some embodiments, the deep learning model is a deep neural network (DNN), a convolutional neural network (CNN), or a kernel ridge regression (KRR). In some embodiments, a CNN is utilized as there is no requirement to define relevant features but are learned from the data sets used for training. In some embodiments, model architectures include hidden layers for nonlinear processing and extraction of important features. In some embodiments, a deep CNN with ResNet-50 architecture is utilized (for more on ResNet, see K. He., et al., arXiv:1512.03385v1 [cs.CV] (2015), the disclosure of which is incorporated herein by reference).

In certain embodiments, the medical disorder to be assessed is Alzheimer's disease and the biomarker to be predicted is accumulation of amyloid beta protein. In certain instances, amyloid beta protein amount is quantified by the standardized uptake value ratio (SUVR), which is determined by positron emission tomography (PET) and the amyloid radiotracer 18F-AV45 (florbetapir). In these embodiments, the deep learning model assesses SUVR of an amyloid PET image acquired at a baseline (t=0) to yield features to predict change of SUVR at a future time point (for more on SUVR feature assessment, see Exemplary Embodiments and F. Reith, et al., Am J Neuroradiol. 41: 980-986 (2020), the disclosure of which is incorporated herein by reference).

As depicted in FIG. 1, a predictive computational model is trained (105) to predict future biomarkers from a set of one or more baseline images. In some embodiments, a model is utilized to predict the change of biomarkers from the baseline image to a future time point.

In some embodiments, a prediction model is trained utilizing biomedical image data. In some embodiments, a prediction model is trained utilizing biomedical image data acquired at baseline and later time points showing progression of the biomarker. In some embodiments, a prediction model is further trained with clinical data, genetic data, or other biomedical data that is relevant to the progression of the medical disorder order.

Any appropriate predictive computational learning model can be utilized, including (but not limited to) linear regression (e.g., LASSO) and gradient-boosted random forest techniques (e.g., gradient-boosted decision trees). Likewise, any appropriate model architecture can be utilized that provides an ability to predict future biomarkers. In some embodiments, no supervision is provided to train the model.

In certain embodiments, the medical disorder to be assessed is Alzheimer's disease and the biomarker to be predicted is accumulation of amyloid beta protein. In certain instances, amyloid beta protein amount is quantified by the standardized uptake value ratio (SUVR), which is determined by positron emission tomography (PET) and the amyloid radiotracer 18F-AV45 (florbetapir). In these embodiments, a predictive computational model is trained to predict change of SUVR from a set of one or more baseline images to a future time point (for more on ΔSUVR prediction, see Exemplary Embodiments). In certain embodiments, a ΔSUVR prediction model incorporates clinical data, genetic data, or other biomedical data, including (but not limited to) age, sex, weight, baseline cognitive testing scores, and apolipoprotein E gene status. Cognitive tests include (but are not limited to) the mini-mental state examination (MMSE) and the Functional Activities Questionnaire (FAQ) total score.

Process 100 also optionally assesses (107) the predictive ability of the trained machine learning model. Accordingly, in some embodiments, trained models are evaluated for their predictive performance utilizing baseline image data of a cohort of subjects. In some embodiments, the baseline image data of the assessment cohort was not utilized in training or validating the model. In some embodiments, the predictive computational learning model performance is assessed via root mean squared error (RMSE). In some embodiments, the predictive computational learning model performance is assessed via cross validation. In some embodiments, the statistical significance of the architecture of the predictive computational model is analyzed via a linear mixed effects model.

While specific examples of processes for training and assessing a predictive model utilizing medical images are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for training and assessing a predictive model appropriate to the requirements of a given application can be utilized in accordance with various embodiments.

### Predicting Biomarker Progression

Several embodiments are directed toward methods of utilizing a trained predictive model to predict biomarker progression, which can be utilized as a diagnostic. In some embodiments, a predictive model can predict the development of a biomarker at a future time point in a subject. In some embodiments, a predictive model can predict the rate a biomarker develops over time in a subject. Accordingly, a subject can be predicted to be a particular progressor type. In some embodiments, a subject is predicted to be a fast progressor, a moderate progressor, or a slow progressor. In some embodiments, the rate of biomarker develops is utilized to inform treatment options. In some embodiments, the rate of biomarker development is utilized to identify certain groups of subjects for a clinical trial. For example, in some instances it is desirable to identify fast progressors for a clinical trial involving dementia and/or Alzheimer's disease such that results of the trial can be concluded more quickly. Furthermore, the number of subjects necessary for enrollment can be decreased as identified fast progressors are ideal candidates, which can be difficult to ascertain by traditional selection criteria.

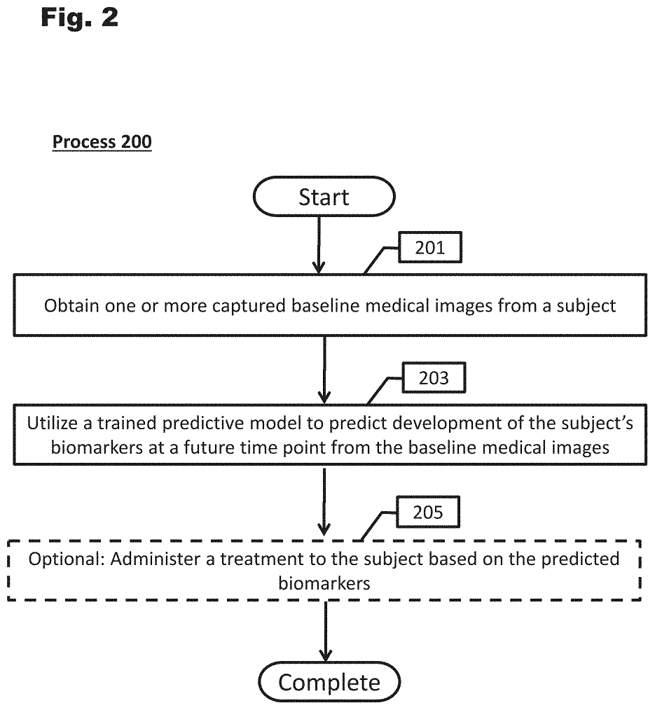

Provided in FIG. 2 is a method for predicting a subject's biomarkers at a future time point from baseline medical images, in accordance with various embodiments. In some embodiments, a predictive model is utilized as a diagnostic. In some embodiments, a predictive model is utilized to identify subjects for a clinical trial. In some embodiments, a predictive model is utilized to determine a treatment for a subject.

Process 200 begins by obtaining (201) a set of one or more captured baseline biomedical images from a subject. Any type of baseline medical images can be obtained as consistent with the disorder pathology and the type of baseline biomedical images utilized in the trained and validated predictive model. Accordingly, baseline biomedical images can be obtained utilizing MRI, X-ray, CT, US, or PET.

In certain embodiments, the medical disorder to be assessed is Alzheimer's disease and the biomarker to be predicted is accumulation of amyloid beta protein. In certain instances, amyloid beta protein amount is quantified by the standardized uptake value ratio (SUVR), which is determined by positron emission tomography (PET) and the amyloid radiotracer 18F-AV45 (florbetapir).

The obtained set of baseline medical images is utilized (203) within a trained predictive model to predict development of the subject's biomarkers at a future time point. Any appropriate trained predictive model or combination of predictive models can be utilized including (but not limited to) linear regression (e.g., LASSO) and gradient-boosted random forest techniques (e.g., gradient-boosted decision trees). In some embodiments, a deep learning model is utilized to identify biomarker features from baseline images. In some embodiments, the deep learning model is a deep neural network (DNN), a convolutional neural network (CNN), or a kernel ridge regression (KRR). In some embodiments, a model is trained and validated as shown in FIG. 1 or described in the examples provided within the Exemplary Embodiments.

In certain embodiments, the subject is assessed for Alzheimer's disease and the accumulation of amyloid beta protein. In certain instances, amyloid beta protein amount is quantified by the standardized uptake value ratio (SUVR), which is determined by positron emission tomography (PET) and the amyloid radiotracer 18F-AV45 (florbetapir). In these embodiments, the predictive computational model is trained to predict change of SUVR from the subject's set of one or more baseline images to a future time point.

Process 200 also optionally administers (205) a treatment to the subject based on the predicted biomarkers. Any appropriate treatment for the disorder assessed can administered. In some embodiments, the treatment is a drug (e.g., small molecule or biologic). In some embodiments, the treatment is a surgical procedure. In some embodiments, the treatment is a prosthetic implant. In some embodiments, the treatment is a vaccine. In some embodiments, the treatment is an experimental treatment, which can be assessed within a clinical trial.

In some embodiments, the subject is placed into a clinical trial based on the predicted progression of the biomarkers. In various embodiments, the subject is predicted to be a fast progressor, a moderate, or a slow progressor. Accordingly, the subject is administered an experimental treatment that is being assessed in the clinical trial.

In certain embodiments, the subject is placed into a clinical trial for an Alzheimer's disease treatment based on the predicted change of SUVR. Accordingly, the subject is administered the experimental Alzheimer's disease treatment that is being assessed in the clinical trial. In some embodiments, the subject is predicted to be a fast progressor, a moderate progressor, or a slow progressor. In some embodiments, the predicted biomarker progression of an individual is utilized to determine selection into and/or placement within a clinical trial.

### Systems for Prediction of Biomarker Progression

A computational processing system to predict biomarker progression in accordance with various embodiments of the disclosure typically utilizes a processing system including one or more of a CPU, GPU and/or neural processing engine. In a number of embodiments, captured image data is processed using an Image Signal Processor and then the acquired image data is analyzed using one or more machine learning models implemented using a CPU, a GPU and/or a neural processing engine. In some embodiments, the computational processing system is housed within a computing device associated with the imaging modality. In some embodiments, the computational processing system is housed separately from and receives the acquired images. In certain embodiments, the computational processing system is in communication with the imaging modality. In various embodiments, the processing system communicates with the imaging modality by any appropriate means (e.g., a wireless connection, hardwired connection, Bluetooth, WiFi, cellular data, etc.). In certain embodiments, the computational processing system is implemented as a software application on a computing device such as (but not limited to) computer, mobile phone, a tablet computer, and/or a wearable device (e.g., watch).

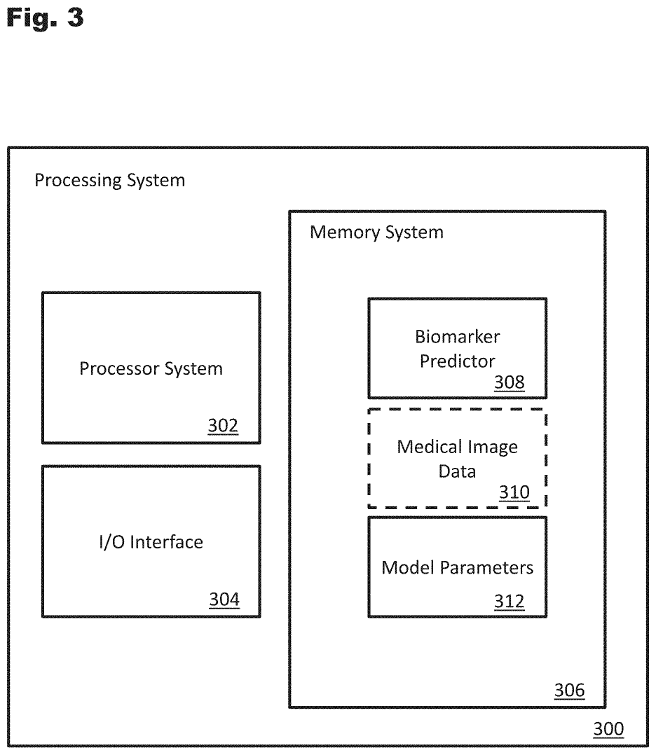

A computational processing system in accordance with various embodiments of the disclosure is illustrated in FIG. 3. The computational processing system 300 includes a processor system 302, an I/O interface 304, and a memory system 306. As can readily be appreciated, the processor system 302, I/O interface 304, and memory system 306 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a biomarker predictor application 308. The biomarker predictor application can be downloaded and/or stored in non-volatile memory. When executed, the biomarker predictor application is capable of configuring the processing system to implement computational processes including (but not limited to) the computational processes described above and/or combinations and/or modified versions of the computational processes described above. In several embodiments, the biomarker predictor application 308 utilizes medical image data 310, which can be stored in the memory system, to perform image processing and predicting of biomarkers from baseline images. In certain embodiments, the biomarker predictor application 308 utilizes model parameters 312 stored in memory to process acquired image data using machine learning models to perform processes including (but not limited to) predicting biomarkers. Model parameters 312 for any of a variety of machine learning models including (but not limited to) the various machine learning models described above can be utilized by the biomarker predictor application. In several embodiments, the medical image data 310 is temporarily stored in the memory system during processing and/or saved for use in training/retraining of model parameters.

While specific computational processing systems are described above with reference to FIG. 3, it should be readily appreciated that computational processes and/or other processes utilized in the provision of biomarker prediction in accordance with various embodiments of the disclosure can be implemented on any of a variety of processing devices including combinations of processing devices. Accordingly, computational devices in accordance with embodiments of the disclosure should be understood as not limited to specific imaging systems, computational processing systems, and/or parametric map generator systems. Computational devices can be implemented using any of the combinations of systems described herein and/or modified versions of the systems described herein to perform the processes, combinations of processes, and/or modified versions of the processes described herein.

### Exemplary Embodiments

The embodiments of the disclosure will be better understood with the various examples provided within. Described in the attached manuscript are examples of how to predict future quantitative standardized uptake value ratio (SUVR), an established biomarker of brain amyloid deposition in Alzheimer's disease. Prediction of future image biomarker is useful in various applications, such as (for example) better targeting of treatments or enrolling patients in a clinical trial.

### EXAMPLE 1

### Predicting Future Amyloid Biomarkers in Dementia Patients with Machine Learning to Improve Clinical Trial Patient Selection

### Methods

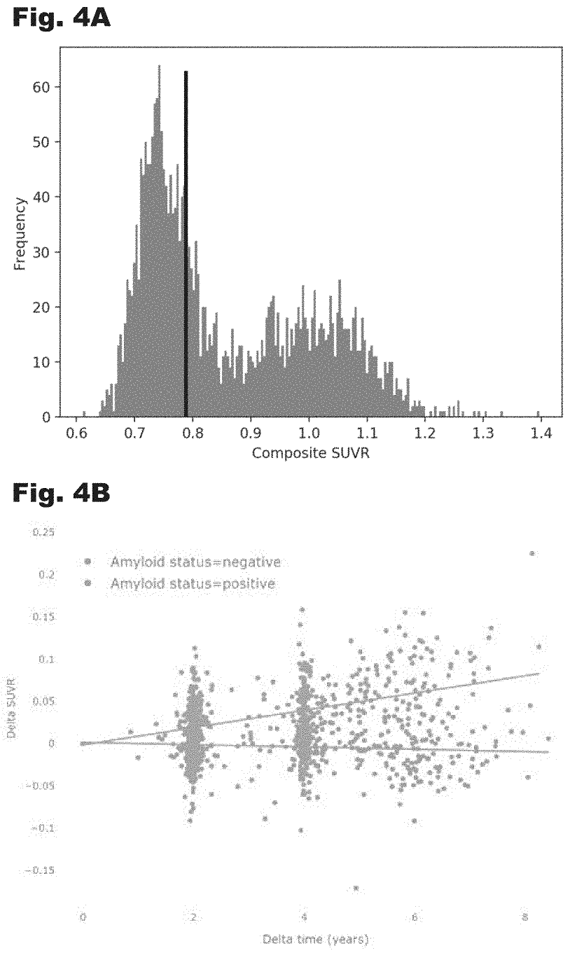

Imaging Information: All available 18F-AV45 (florbetapir, Avid Lilly, Philadelphia, Pa.) PET studies from ADNI as of August 2019 were obtained. All scans were downloaded in Neuroimaging Informatics Technology Initiative (NIFTI) file format along with the UC Berkeley AV45 analysis to obtain a standardized uptake value ratio (SUVR) values based on a reference region consisting of cerebellum, brainstem/pons, and eroded white matter (SUMMARYSUVR_COMPOSITE_REFNORM). Higher SUVR reflects more amyloid deposition in supratentorial cortical regions. Baseline SUVR distribution is shown in FIG. 4A. All patients with multiple scans were selected and the interval SUVR change from baseline (ΔSUVR) were calculated (FIG. 4B).

An aim of this study is to predict the SUVR change on images taken after the baseline scan. The first scan of a subject is assigned a delta time (ΔT)=0, while a scan 2 years later is assigned ΔT=2. Similarly, the baseline SUVR is defined as SUVR_t0, and it is subtracted from all their later scans to calculate ΔSUVR, which represents the target of the prediction. To estimate the future SUVR, ΔSUVR is added to SUVR_t0.

Clinical and Genetic Information: For model development, several clinical and genetic features, including patient age, sex, weight, baseline cognitive testing scores, and apolipoprotein E (APOE) gene status were also included. Two cognitive tests were included: the mini-mental state examination (MMSE) and the Functional Activities Questionnaire (FAQ) total score. The polymorphic expression of the APOE gene was also included, as the APOE genotype is known to strongly affect amyloid deposition. To assess performance of the model in different clinical cohorts, we examined clinical status using the clinical dementia rating (CDR) score if it was made ±50 days of the baseline PET scan.

Prediction Model—Linear Regression: Multivariate regression using the StatsModels library in Python was performed, which fits the following equation:

$${y}_{i} = {β}_{0} constant + {β}_{1} {x}_{i 1} + {β}_{1} {x}_{i 2} + … + {β}_{n} {x}_{i n}$$

Based on multivariate regression, the significance p-value was calculated for each independent variable.

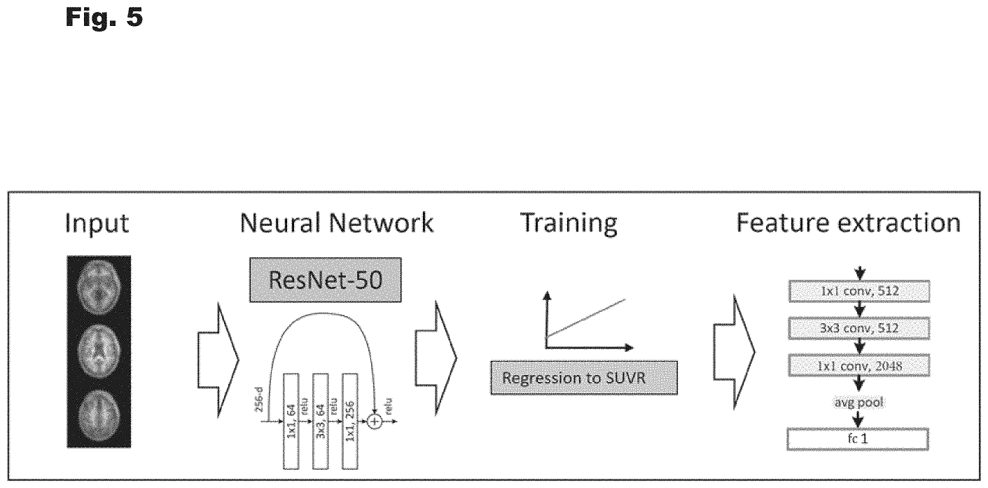

Prediction Model—Deep Learning: A convolutional neural network (CNN) was trained to predict amyloid PET SUVR, using methods described in Reith. (See e.g., Reith F, et al. Alzheimer's Disease Neuroimaging Initiative. Application of Deep Learning to Predict Standardized Uptake Value Ratio and Amyloid Status on 18F-Florbetapir PET Using ADNI Data. AJNR Am J Neuroradiol. 2020; 41: 980-986; the disclosure of which is hereby incorporated by reference in its entirety.) Of note, the CNN is not trained to predict future SUVR change, but instead learned image features associated baseline SUVR. In brief, the ResNet-50 architecture was used. Network input was three centrally located slices. Standard ResNet ends with a layer for distinguishing 1000 differing classes, but it was modified for SUVR prediction (a regression task). The final layer was changed to a single output without an activation function. The cost function was the mean squared error between predicted and true SUVR using the ADAM optimizer. The best-performing hyperparameters was applied for training on current SUVR and settled on an initial learning rate of 0.0001, 30 epochs, with 10× decrease of learning rate every 10 epochs. Training time was 22 min. The model was pre-trained using the ImageNet dataset of natural images. After training, PyTorch was used to extract the last layer's activations. This resulted in 2048 numbers (features) for each individual PET scan (FIG. 5).

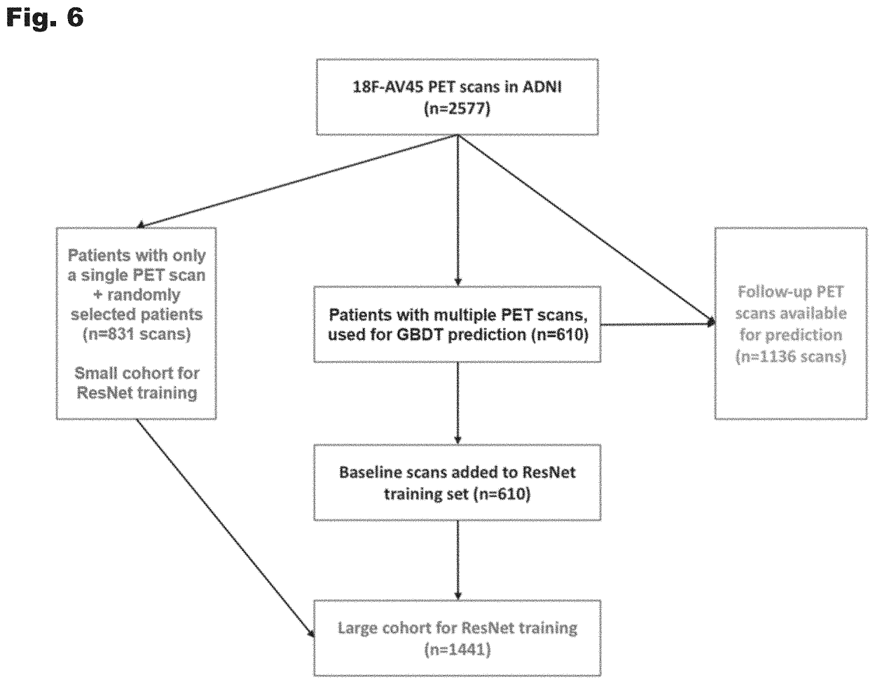

Since the goal was to predict SUVR change based on baseline patient information and the ResNet-50-derived features, this network was trained on baseline images only. The training set consisted of 1441 amyloid PET scans (1099 baseline scans and 342 follow-up scans). A cross-validation testing design (described later) was used such that none of the follow-up scans used for training were from patients that were evaluated for ΔSUVR in the test set. A smaller training dataset consisting of 831 baseline scans was also tested to demonstrate the effect of larger training sets and the details and results are found in the Supplemental Materials. The test set consisted of all follow-up amyloid PET scans (n=1136 scans in 610 subjects) (FIG. 6).

Prediction Model—Gradient Boosting Decision Tree: To combine clinical/genetic features and deep imaging features, a gradient boosting decision tree (GBDT) algorithm was used , specifically the LightGBM implementation, to predict SUVR change. Regression was defined as the objective of the GBDT and optimized through a mean squared error loss function. The goal is to predict on average results with the lowest root mean squared error (RMSE) with respect to the true ΔSUVR value, so lower RMSE values reflect better performance.

GBDT models were tested with and without deep learning-based PET features to assess the importance of the images. The models were trained for 4000 iterations creating a total of 4000 DTs. To prevent overfitting, each DT was created with a bagging fraction of 0-5. During training, for this example, a minimum of 9 samples was required for each DT leaf created. The maximum number of DT leaves is set to 50. A GBDT model incorporating clinical features was compared with a GBDT model that incorporates clinical features as well as the PET scan activations from ResNet. There are 8 clinical features and 2048 image-based deep ResNet activations. When predicting based on clinical features only, in this example, the DT's depth was limited to 4 and set the feature fraction and learning rate to 80% and 0.0006, respectively. When ResNet activations are included, the DT's depth was set to 9, the feature fraction to 50%, and the learning rate to 0.0045. An informed grid search was performed, testing multiple hyperparameters for GBDT with and without activations. GBDT with activations took significantly longer to train (256 s vs 13 s for GBDT without activations).

When feature activations were used, three types of activations were compared. One type consisted simply of random numbers that objectively do not contain any information at all, reflecting no impact of the imaging features. These random numbers were drawn from a normal distribution consistent with the mean and variance of the ResNet features themselves. The other two are based on the two ResNet trainings. One based on 831 data points (referred to as “n=831 ResNet activations”), the others based on 1441 PET scans (referred to as “n=1441 ResNet activations”). As the best results were achieved based on the latter, GBDT with activations refers to ResNet activations based on a training set of 1441 PET scans, if not otherwise specified.

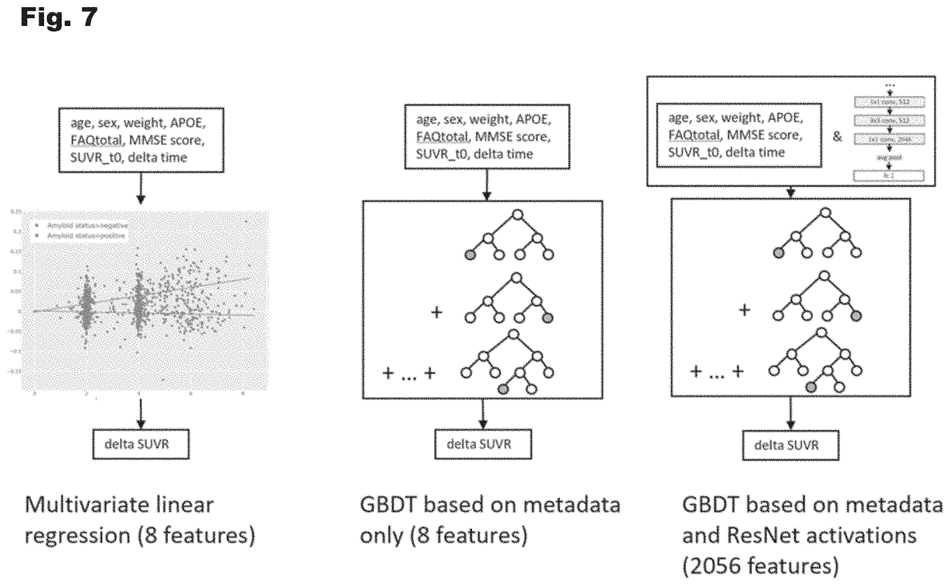

A summary of the models constructed and tested is provided in FIG. 7: Multivariate linear regression (8 input features), GBDT based on the 8 clinical features only, GBDT based on clinical features and ResNet activations (8+2048 input features).

Data Analysis: GBDT performance was analyzed via root mean squared error (RMSE) for ΔSUVR in all follow-up scans (1136 scans in 610 individuals). Five-fold cross validation was performed to present the average RMSE. For cross validation purposes, the dataset was divided into five distinct parts. Each part had its unique subjects, meaning no subject can be found in more than one of the five distinct parts, guaranteeing that there is no subject shared by both the training and test sets. The statistical significance of model design choices was analyzed with linear mixed effects models and Wilcoxon rank sum tests, as appropriate. For these measures, the squared error of ML system predictions were compared.

To assess the practical value of these SUVR predictions, these predictions were also used to select subjects with the highest SUVR changes. The rationale is that these patients might be desirable candidates for clinical trials assessing the impact of an amyloid lowering agent. The top 10% of cases (61 individuals) with the highest ΔSUVR were identified. In subjects with multiple follow-up scans, the scan with the maximum ΔSUVR was selected. The performance of multivariate linear regression was assessed, GBDT without imaging features, and GBDT with imaging features by calculating the % of these top progressors also predicted by the model. For example, a random selection would lead to a 10% “hit rate,” while the models should be able to improve upon this if they are making more accurate predictions.

Model performance were compared with other methods of selection in two ways. The first is to randomly select patients that meet a specific criterion. The following groups for these tests were included: amyloid positive at baseline (n=313), presence of at least one APOE ε4 allele (n=237), mildly positive amyloid patients (defined as baseline SUVR between 0.79 and 0.95) (n=156), amyloid positive with at least one APOE ε4 allele (n=178), and mildly amyloid positive subjects with at least one APOE ε4 allele (n=70). The various models' performance in pre-selected groups often targeted in clinical trials were also examined, specifically: mildly positive amyloid patients (as defined above) and subjects with mild dementia (baseline CDR 0.5) (n=229). Since these latter datasets start from a smaller denominator, the task was to identify the top 20% fastest true ΔSUVR progressors.

Results

Patient Cohort: The baseline demographics and clinical features of the 610 unique subjects with 1136 follow-up scans are summarized in Table 1. The time horizon of the follow-up predictions was as follows: 1-3 years (n=553), 3-5 years (n=354), and 5+ years (n=227).

ResNet-50 training and feature extraction: For ResNet-50 image features training on 831 samples, amyloid status prediction (positive versus negative, defined as SUVR less than or greater than 0.79) was correct on 97.5% on the train set and 89.7% on the test set. On the larger sample size of 1441 samples, train and test accuracy were 98.1% and 93.7%, respectively. This showed that the ResNet feature training pipeline successfully identifies features related to SUVR.

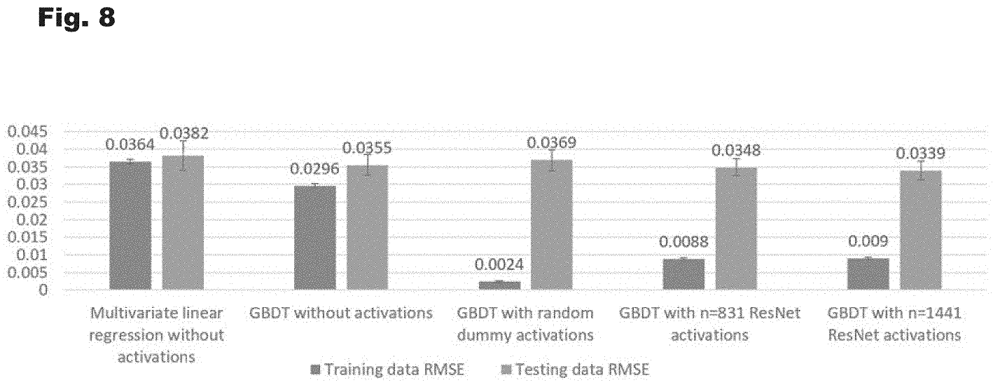

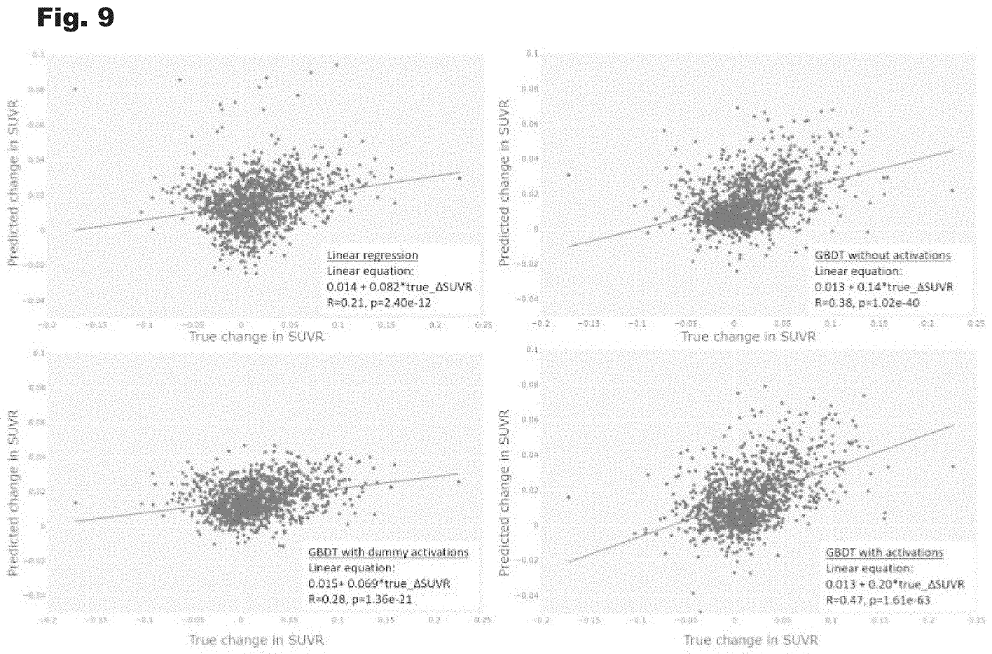

ΔSUVR Prediction: Visual presentation of the performance of each model is shown in FIG. 8. For multivariate linear regression, an average RMSE of 0.0364±0.0007 and 0.0382±0.004 was found on the training and test sets, respectively. The weights and significance of each feature are shown in Table 2. In contrast, using the GBDT with the same inputs (8 clinical features) without deep activations, slightly better performance was found (RMSE 0.0296±0.0007 train, 0.0355±0.0003 test). Using random activations in place of the deep activations, the model heavily overfits the training set (0.0024±0.0003) but had worse performance on the test set (0.0369±0.0003). For the GBDT model incorporating the deep imaging activations, the best performance was identified (0.0090±0.0004 train, 0.0339±0.0003 test). While the performance difference between train and test for the GBDT with imaging activations suggests residual overfitting, applying regularization did not improve test set performance, and of course, the ultimate proof of their superiority is their better performance on the held-out test set. There was significantly better performance of the GBDT model with activations compared with the GBDT model that used dummy activations (p<2.35e-9) or the GBDT model that only used the 8 clinical features (p<0.00388), measured using the Wilcoxon rank sum test. The correlation between ΔSUVR predictions and ground truth changes were additionally compared (FIG. 9). Similar to the findings of RMSE, the worst performing method is linear regression (R=0.21), while the best performing method is GBDT with deep activations (R=0.47).

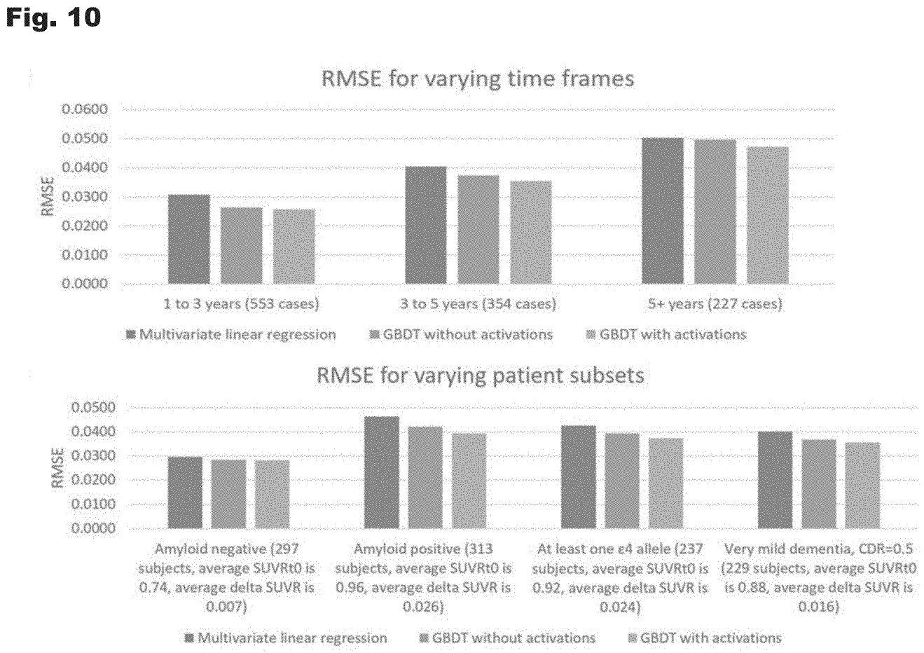

Model performance was analyzed for the various time horizons of prediction (FIG. 10). Two follow-up scans were excluded, as they were performed less than a year from baseline. In the shortest timeframe (1-3 years), accuracy was highest for all ML algorithms. This performance decreases slightly in the 3-5 year and 5+ year time frames. In all cases, GBDT with activations performed best.

GDBT with activations also performed better in many different subsets of the full cohort used in clinical trials. Results for initially amyloid negative patients, amyloid positive patients, patients with at least one APOE ε4 allele, and patients with mild cognitive impairment (CDR 0.5) are shown in FIG. 10. In initially amyloid negative patients, performance was similar between the different models, but for the other groups, the GBDT model with activations performed better.

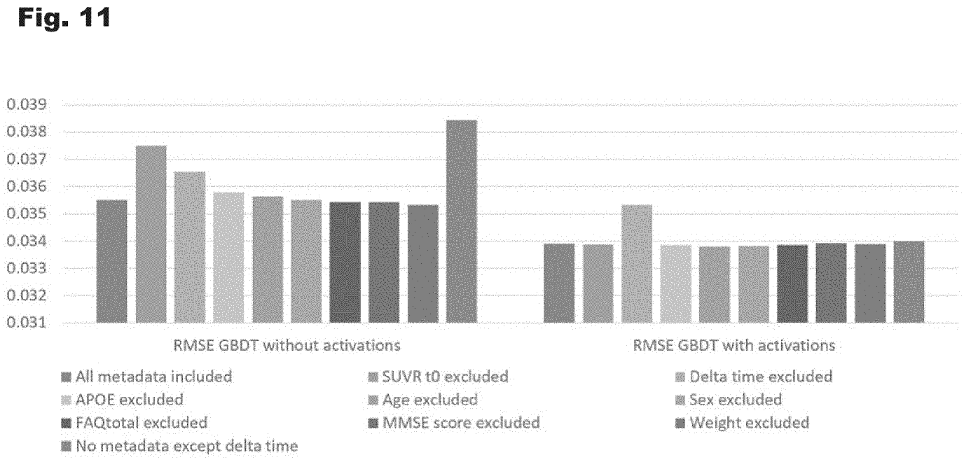

The importance of various individual features was explored by removing individual features and measuring the effect on RMSE. In general, for models without activations, removing baseline SUVR and delta time made the biggest difference. When deep activations were used, only delta time omission led to a significant degradation in prediction performance (FIG. 11).

The biggest effect on prediction accuracy has the omission of SUVR t0. When this value is omitted, it was found that performance decreases from 0.0355 to 0.0375. Another significant decrease is seen when removing delta time (RMSE of 0.0365). The other features had smaller effects on GBDT without activations performance. Removing weight from input features actually slightly improves performance, allowing GBDT without activations to reach a RMSE of 0.0353. Looking at GBDT without activations, the results identify SUVR t0, delta time, and APOE as the top features in terms of relevancy.

For GBDT with activations, the only significant drop in performance is seen when delta time is removed, with RMSE increasing from 0.0339 to 0.0353. Removing any of the other individual clinical features did not change performance significantly, with RMSE ranging between 0.0338 and 0.0339.

When looking at GBDT with activations after removing all clinical metadata except for delta time, a RMSE of 0.0340 was achieved, still surpassing GBDT without any ResNet activations.

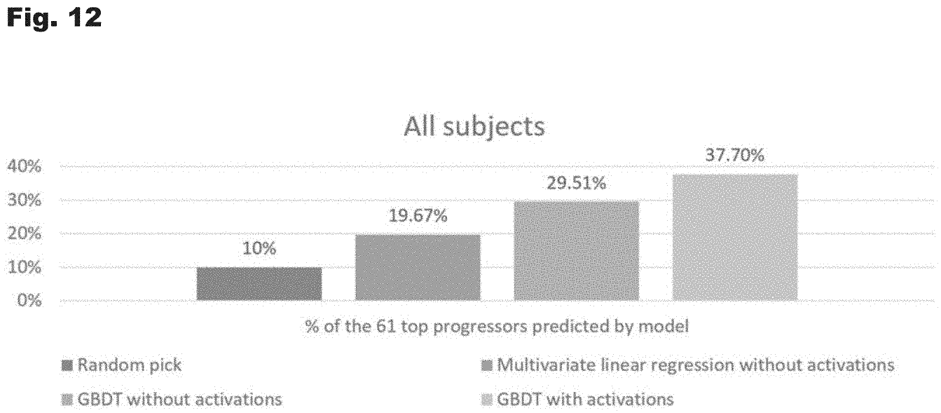

Implications for Study Selection: The ability of the various models to identify the fastest 10% of true amyloid accumulators were evaluated (FIG. 12). When selecting using linear regression, it was found that 19.7% of this group are also in the top 10% of ground truth patients. This is already twice the number of subjects compared to the expected number of subjects selected via random pick. Selection based on GBDT without activations led to 29.5% identification. Highest performance was obtained using GBDT with activations, with 37.7% of the fastest progressors identified, an almost 4× increase in yield compared with a random selection. Sensitivity and specificity tables along with positive and negative likelihood ratios for each model in these cohorts are included in the Table 3.

These results were compared to other ways of selecting fast progressors using the entire cohort. By randomly select baseline amyloid positive subjects, a correct prediction would be made of the top 61 progressors in 16% (50/313 of baseline amyloid positive subjects). Among the other methods of choosing fast progressors (at least one APOE ε4 allele, mildly positive amyloid patients, amyloid positive with at least one APOE ε4 allele, and mildly amyloid positive subjects with at least one APOE ε4 allele), best performance is the last group (25.7%), still significantly lower than the GBDT with activations model.

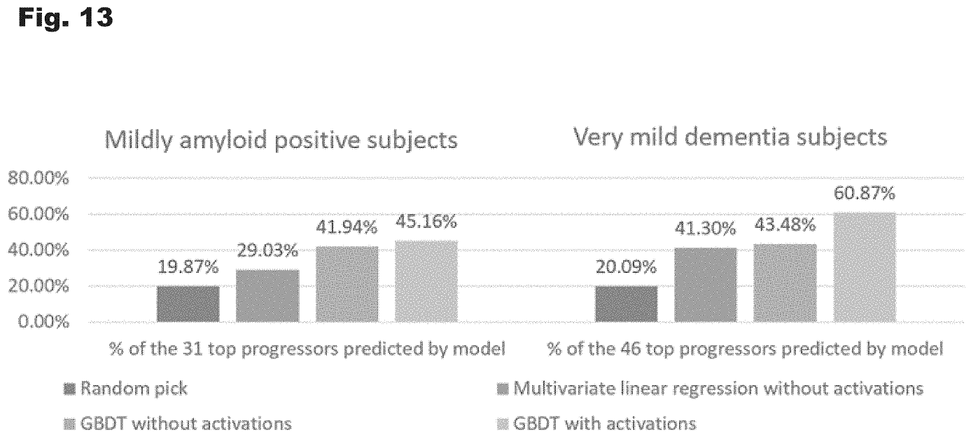

Model performance was examined in clinically relevant subgroups. For mildly positive amyloid patients, of the top 20% fastest progressors, performance increases to 29.0% for linear regression, 41.9% for GBDT without activations, and 45.2% for GBDT with activations. For subjects with mild dementia at baseline (CDR 0.5), performance increases from 41.3% for linear regression, 43.5% for GBDT without activations, and 60.9% for GBDT with activations. These subjects had a baseline SUVR of 0.88 [IQR 0.75, 1.00], similar to that of the subjects identified by GBDT with activations (0.92 [IQR 0.85, 0.97]) (FIG. 13).

DISCUSSION: This study extends prior work at using neural networks to predict current SUVR for amyloid PET studies in the ADNI cohort to predict SUVR in the future. This trait is accomplished by training a network on longitudinal studies and by including clinical and genetic features. It was found that a GBDT that includes clinical, demographic, and genetic features combined with deep activations created from ResNet-50 had the best performance. This study showed the value of this quantitatively, measuring the mean error in the prediction of the SUVR change, as well as on a practical basis, showing that using this approach can identify the fastest amyloid accumulators in both the entire test dataset as well as in clinically relevant sub-populations at a 2-4× higher rate than random selection or other commonly used selection methods. This latter capability might be useful to enrich research studies that target this biomarker, such as an amyloid-clearing pharmacological agent, reducing costs and speeding up clinical trials. Fundamentally, the idea of using deep learning to combine imaging and clinical information with the goal of predicting future imaging biomarkers, including its possible use in patients receiving different treatments, could be a fruitful pathway towards more personalized medicine.

This study shows that adding deep features to the GBDT improves performance for both RMSE and selection of fast progressors and further shows that performance improves by obtaining better deep features by training on a larger number of PET scans (e.g., 1441 vs. 831 subjects), strongly suggesting that the model is learning relevant features for this prediction task. It also highlights the value of large shared datasets such as ADNI for ML methods using deep learning feature identification. The study also found that the use of deep features make the model less dependent on missing clinical, demographic, or genetic data as shown by the studies in which individual features are selectively removed. One advantage of combining deep activations with the GBDT structure enabled the evaluation of the role of specific features and how sensitive the models are to missing data.

CONCLUSION: This example trained a machine learning algorithm to combine deep image features with clinical, demographic, and genetic information in order to predict future changes in amyloid deposition. Practically, it was shown to be superior to several other methods of identifying patients to identify fast progressors. This method is adaptable to study other important imaging biomarkers and to assess the effects of different treatments and may have advantages over models trained to predict clinical endpoints.

### Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

TABLE 1
Baseline demographics and SUVR of the 610 patients. Delta time
and ΔSUVR are based on 1136 follow-up data points used
for training and testing the GBDTs. Please note that the CDR
values were not used for model training or testing but used
to compare performance in clinically relevant subgroups.

| Clinical feature | Value, Mean ± SD, (IQR) | |
|---|---|---|
| Age (yrs) | 73.1 ± 7.4 | (67.9, 78.1) |
| Sex | 46.4% female, 53.6% male | |
| Weight (kg) | 78.3 ± 15.8 | (68.0, 87.0) |
| APOE | ε2/ε2 0.2%, ε2/ε3 9.7%, ε3/ε3 49.3%, ε2/ε4 | |
| | 1.9%, ε3/ε4 32.1%, ε4/ε4 6.7%. | |
| FAQtotal | 2.4 ± 4.7 | (0, 2.0) |
| MMSE | 27.4 ± 3.4 | (26.0, 30.0) |
| Baseline SUVR | 0.84 ± 0.14 | (0.74, 0.96) |
| CDR | CDR: 39.9%, 0.5 CDR: 55.1%, 1 CDR: 4.1%, | |
| | 2 CDR: 0.7%, 2 CDR: 0.3% | |
| Delta time (yrs) | 3.5 ± 1.6 | (2.0, 4.3) |
| ΔSUVR | 0.016 ± 0.038 | (−0.0085, 0.037) |

TABLE 2
Weight, standard error, and p-value of each multivariate
linear regression feature. Results are from one of the
five cross-validation folds. Input features are normalized
before being fed to the multivariate linear regression.

| | | Standard | | |
|---|---|---|---|---|
| Feature | Coefficient | error | p-value | IQR of coefficient |
| Baseline SUVR | 0.028 | 0.009 | 0.003 | [0.01, 0.046] |
| Sex | 0.006 | 0.003 | 0.017 | [0.001, 0.012] |
| Weight | 0.004 | 0.007 | 0.54 | [−0.009, 0.017] |
| Delta time | 0.015 | 0.003 | 0 | [0.009, 0.02] |
| MMSE | −0.02 | 0.025 | 0.417 | [−0.07, 0.029] |
| FAQtotal | −0.003 | 0.001 | 0 | [−0.004, −0.001] |
| Age | 0.055 | 0.013 | 0 | [0.029, 0.081] |
| APOE ε2/ε2 | −0.087 | 0.041 | 0.032 | [−0.167, −0.008] |
| APOE ε2/ε3 | −0.083 | 0.031 | 0.007 | [−0.143, −0.023] |
| APOE ε3/ε3 | −0.068 | 0.03 | 0.026 | [−0.128, −0.008] |
| APOE ε2/ε4 | −0.07 | 0.032 | 0.031 | [−0.133, −0.006] |
| APOE ε3/ε2 | −0.063 | 0.031 | 0.04 | [−0.123, −0.003] |
| APOE ε4/ε4 | −0.049 | 0.031 | 0.112 | [−0.11, 0.011] |

TABLE 3
Sensitivity, specificity, and likelihood ratios for different models.

| | | True ΔSUVR | | |
|---|---|---|---|---|
| Linear | | Absent | Present | |
| Regression | | (lower 90%) | (upper 10%) | Totals |
| Predicted | Predicted top 10% | 49 | 12 | 61 |
| ΔSUVR | Predicted lower 90% | 500 | 49 | 549 |
| | Totals | 549 | 61 | 610 |

Sensitivity: 0.20 (95% confidence interval: 0.11-0.32); Specificity: 0.91 (0.88-0.93)
Positive likelihood ratio: 2.2 (1.2-3.9); Negative likelihood ratio 0.9 (0.8-1.0)

| | | True ΔSUVR | | |
|---|---|---|---|---|
| GBDT without | | Absent | Present | |
| deep activations | | (lower 90%) | (upper 10%) | Totals |
| Predicted | Predicted top 10% | 43 | 18 | 61 |
| ΔSUVR | Predicted lower 90% | 506 | 43 | 549 |
| | Totals | 549 | 61 | 610 |

Sensitivity: 0.30 (95% confidence interval: 0.19-0.43); Specificity: 0.92 (0.90-0.94)
Positive likelihood ratio: 3.8 (2.3-6.1); Negative likelihood ratio 0.8 (0.7-0.9)

| | | True ΔSUVR | | |
|---|---|---|---|---|
| GBDT with | | Absent | Present | |
| deep activations | | (lower 90%) | (upper 10%) | Totals |
| Predicted | Predicted top 10% | 38 | 23 | 61 |
| ΔSUVR | Predicted lower 90% | 511 | 38 | 549 |
| | Totals | 549 | 61 | 610 |

Sensitivity: 0.38 (95% confidence intervals: 0.26-0.51); Specificity: 0.93 (0.91-0.95)
Positive likelihood ratio: 5.4 (3.5-8.5); Negative likelihood ratio 0.7 (0.6-0.8)

What is claimed is:

1. A method of predicting future biomarkers, comprising:
obtaining a set of one or more baseline medical images, wherein the set of one or more baseline medical images was captured from a subject, and wherein the set of baseline medical images contains one or more biomarkers that are associated with a medical disorder; and
utilizing a predictive model and the set of baseline biomedical images to predict the progression of the one or more biomarkers.

2. The method as in claim 1, wherein the predictive model was trained with image data of a training cohort of individuals, each individual of the cohort having the medical disorder and the image data comprising baseline images and images taken later time points showing progression of the one or more biomarkers.

3. The method as in claim 2, wherein the prediction model is further trained with one or more clinical data or genetic data features.

4. The method as in claim 3, wherein the one or more clinical or genetic features is selected from: patient age, sex, weight, baseline cognitive testing scores, and apolipoprotein E (APOE) gene status.

5. The method of claim 3 further comprising:
obtaining clinical data or genetic data of the individual; and
utilizing the obtained clinical data or genetic data within the predictive model along with the set of baseline biomedical images to predict the progression of the one or more biomarkers.

6. The method as in claim 1, wherein the predictive model utilizes image features identified from a deep learning computational model.

7. The method as in claim 6, wherein the deep learning computational model incorporates a deep neural network (DNN), a convolutional neural network (CNN), or a kernel ridge regression (KRR).

8. The method as in claim 1, wherein the predictive model incorporates linear regression or a gradient-boosted random forest technique.

9. The method as in claim 1 further comprising:
predicting progressor type of the subject based on the predicted progression of the one or more biomarkers.

10. The method as in claim 9, where in the progressor type is: slow progressor, moderate progressor, or fast progressor.

11. The method as in claim 9 further comprising:
administering a treatment to the subject based on the predicted progression of a biomarker or the predicted progressor type of the subject.

12. The method as in claim 9 further comprising:
administering an experimental treatment to the subject as part of a clinical trial, wherein the subject is enrolled within the clinical train based the predicted progression of the biomarker or the predicted progressor type of the subject.

13. The method as in claim 1, wherein the medical disorder is Alzheimer's disease and the one or more biomarkers comprises accumulation of amyloid beta protein.

14. The method as in claim 1, wherein the medical disorder is Parkinson's disease and the one or more biomarkers comprises accumulation of Lewy bodies.

15. A computational system for predicting biomarkers, comprising:
memory; and
a set of one or more processors; and
an application stored within the memory, wherein the application is a predictive computational model for predicting biomarkers;
wherein the set of one or more processors is capable of performing the steps of the application, wherein the steps comprise:
assess a set of one or more baseline medical images, wherein the set of one or more baseline medical images was captured from a subject, and wherein the set of baseline medical images contains one or more biomarkers that are associated with a medical disorder; and
predict the progression of the one or more biomarkers based on the assessment of one or more baseline medical images.

16. The system of claim 15, wherein the predictive model was trained with image data of a training cohort of individuals, each individual of the cohort having the medical disorder and the image data comprising baseline images and images taken later time points showing progression of the one or more biomarkers.

17. The system of claim 15, wherein the prediction model is further trained with one or more clinical data or genetic data features.

18. The system of claim 17, wherein the one or more clinical or genetic features is selected from: patient age, sex, weight, baseline cognitive testing scores, and apolipoprotein E (APOE) gene status.

19. The system of claim 17, wherein the steps of the application further comprise:
utilizing clinical data or genetic data derived from the subject within the predictive model along with the set of baseline biomedical images to predict the progression of the one or more biomarkers.

20. The system of claim 15, wherein the predictive model utilizes image features identified from a deep learning computational model.

21. The system of claim 20, wherein the deep learning computational model incorporates a deep neural network (DNN), a convolutional neural network (CNN), or a kernel ridge regression (KRR).

22. The system of claim 15, wherein the predictive model incorporates linear regression or a gradient-boosted random forest technique.

23. The system of claim 15 further comprising:
an imaging modality in communication with the set of one or more processors, wherein the imaging modality is capable of capturing the set of one or more baseline medical images.

24. The system of claim 23, wherein the steps of the application further comprise:
capturing the set of one or more baseline medical images from the subject.

25. The system of 23, wherein the imaging modality comprises one or more of: positron emission tomography (PET), computed tomography (CT), magnetic resonance imaging (MRI), X-ray, fluoroscopic imaging, and ultrasound sonography.
